(19) United States
(12) Patent Application Publication
(10) Pub. No.: US 2023/0345970 A1
(43) Pub. Date: Nov. 2, 2023

(54) COMPOSITION, METHODS, AND USES FOR PEA PROTEIN ISOLATES HAVING IMPROVED DISSOLUTION CHARACTERISTICS

(71) Applicant: CORN PRODUCTS DEVELOPMENT, INC, WESTCHESTER, IL (US)

(72) Inventors: Akshay ARORA, Bridgewater, NJ (US); Amanda KUNZ, Bridgewater, NJ (US); Hanyu YANGCHENG, Bridgewater, NJ (US)

(21) Appl. No.: 17/923,037

(22) PCT Filed: May 19, 2021
(86) PCT No.: PCT/US2021/033127

§ 371 Date: Nov. 3, 2022

Related U.S. Application Data
(60) Provisional application No. 63/027,595, filed on May 20, 2020.

(51) Int. Cl. A23L 33/185; A23J 3/14; C07K 14/415; A23J 3/34; A23L 2/66; A23L 2/39

(52) U.S. Cl. CPC A23J 3/346; A23J 3/14; A23L 2/39; A23L 2/66; A23L 33/185; C07K 14/415

(57) ABSTRACT

The technology disclosed in this specification pertains to a deamidated pea protein isolates, processes for making a deamidated pea protein isolate, and use of the deamidated pea protein isolate in food and beverage compositions. The disclose deamidated pea protein isolates have dissolution properties that are significantly improved compared to non-deamidated pea protein isolates. Furthermore, the deamidated protein isolates maintain good dissolution properties throughout storage.

The technology disclosed in this specification pertains to deamidated pea protein isolates and uses of such isolates in food composition.

Isolated proteins are useful food ingredients. They can be used in food compositions (which for convenience in this specification includes beverages compositions) to provide nutrition or to provide functional performance. Proteins selected for their functional performance, commonly suffer from changes in their functional performance over time. On class of functional attributes of interest for protein isolates is their ability to dissolve in aqueous solutions. It has been observed, however, that the dissolution properties of protein-isolates commonly decrease over time, and so the protein isolates become less functional. Methods for preparing protein isolates that can stabilize their functionality over time are desirable.

This specification discloses deamidated pea protein isolates having stable dissolution profiles compared to non-deamidated pea protein isolates. This specification also discloses methods for making deamidated pea protein isolates. This specification also discloses food compositions comprising the disclosed pea protein isolates.

### BRIEF DESCRIPTION OF THE FIGURES

The technology disclosed in this specification can be better understood with reference to the following figures which are not intended to be limiting in any way:

FIG. 1 graphs solubility profiles of various pea protein isolates at different pH.

FIG. 2 graphs the number of particles having size less than 50 microns in various pea protein isolates measured over time in aqueous slurries.

FIG. 3 graphs the number of particles having size greater than 50 microns in various pea protein isolates.

FIG. 4 graphs the number of particles having size between 150 and 300 microns in various pea protein isolates measured over time in aqueous slurries.

FIG. 5 graphs the relative dispersibility of various pea protein isolate after storage.

FIG. 6 graphs differences in sensory perception of slurries using various pea protein isolates.

FIG. 7 graphs differences in sensory perception of slurries using various pea protein isolates.

In one aspect, the technology disclosed in this specification pertains to a deamidated pea protein isolate. Pea protein exists in peas in amounts between about 20% and about 30% by weight of the pea. The protein can be isolated to obtain pea protein isolates having about 70%, or greater than about 75%, or from about 75% to about 90% or from about 75% to about 85% protein by weight. The rest of the pea protein isolate is comprised substantially of pea starch and pea fiber (cellulosic material). Pea protein isolates useful for any embodiment of the deamidated pea protein isolate described in this specification may be obtained from any known method.

In any embodiment described in this specification, a deamidated pea protein isolate has a degree of deamidation of between about 1% and about 99%, or about 1% and about 90% or about 1% and about 80%, or about 1% and about 70%, or about 1% and about 60%, or about 1% and about 50%, or about 1% and about 40% or about 1% and about 30%. In any embodiment described in this specification, a deamidated pea protein isolate has a degree of deamidation of between about 5% and about 99%, or about 5% and about 90% or about 5% and about 80%, or about 5% and about 70%, or about 5% and about 60%, or about 5% and about 50%, or about 5% and about 40% or about 5% and about 30%. In any embodiment described in this specification, a deamidated pea protein isolate as a degree of deamidation of between about 10% and about 25%, or between about 15% and about 25%, or between 20% and 25%. In any embodiment described in this specification, a pea protein isolate has a degree of deamidation of between about 10% and about 20%, or about 12% to about 20% or about 15% to about 20%. In any embodiment described in this specification, a deamidated pea protein isolate has a degree of deamidation of between about 10% and about 15%, or about 12% and about 14%. In any embodiment described in this specification, a deamidated pea protein isolate has a degree of deamidation of between about 17% and about 23%, or between about 19% and about 21%. In any embodiment described in this specification, a deamidated pea protein isolate has a degree of deamidation of between about 22% and about 25%, or between about 22% and 24%. In any embodiment described in this specification, a deamidated pea protein isolate has a degree of deamidation of between about 10% and about 20% or about 12% and 20%, or from about 15% to about 20%. In any embodiment described in this specification, a deamidated pea protein isolate has a degree of deamidation of 10% and 17% %, or about 12% to less than 17%, or about 12% to about 16%.

In any embodiment described in this specification a deamidated pea protein isolate comprises a soluble material content. In any embodiment described in this specification, a pea protein isolate has a soluble material content of between about 20% and about 40%, or about 20% and about 35%, or about 20% and about 30%. In any embodiment described in this specification, the amount of a soluble material content in a deamidated pea protein varies depending on the degree of deamidation. In any embodiment described in this specification, the amount of soluble material content in a deamidated pea protein isolate varies depending on the pH of the solvent.

In any embodiment described in this specification, a deamidated pea protein isolate has a percent soluble material content at pH 6 of from about 20% to about 30%, or about 21% and about 27% (wt%). In any embodiment described in this specification, a deamidated pea protein isolate has a percent soluble material content at pH 6 of between about 20% and about 25%, or between about 20% and about 24%, or between about 20% and about 23% (wt%). In any embodiment described in this specification, a deamidated pea protein isolate has a percent soluble material content at pH 6 of between about 21% and about 24%, or about 22% and about 24% (wt%). In any embodiment described in this specification, a deamidated pea protein isolate has a percent soluble material content at pH 6 of between about 22% and 26%, or about 24% and about 26% (wt%). In any embodiment described in this specification, a deamidated pea protein isolate has a percent soluble material content at pH 6 of between about 25% and about 30%, or about 26% and about 28% (wt%).

In any embodiment described in this specification, a deamidated pea protein isolate has a percent soluble material content at pH 2 of from about 15% to about 30%, or from about 17% to about 28%. In any embodiment described in this specification, a deamidated pea protein isolate has a percent soluble material content at pH 2 of between about 15% and about 20%, or about 17% and 20% (wt%). In any embodiment described in this specification, a deamidated pea protein isolate has a percent soluble material content at pH 2 of between about 18% and about 24%, or about 18% to about 22% (wt%). In any embodiment described in this specification, a deamidated pea protein isolate has a percent soluble material content at pH 2 of between about 22% and 26%, or about 24% and about 26% (wt%). In any embodiment described in this specification, a deamidated pea protein isolate has a percent soluble material content at pH 2 of between about 25% and about 30%, or about 26% and about 28% (wt%).

In any embodiment described in this specification, a deamidated pea protein isolate has a percent soluble material content at about pH 7 of from about 30% to about 40%, or from about 34% to about 38% (wt%). In any embodiment described in this specification, a deamidated pea protein isolate has a percent soluble material content at about pH 7 of between about 30% and about 36%, or about 33% and about 36 (wt%). In any embodiment described in this specification, a deamidated pea protein isolate has a percent soluble material content at about pH 7 of between about 35% and about 40%, or between about 35% and 37% (wt%).

In any embodiment described in this specification, a deamidated pea protein isolate has a median particle size of between 75 and 125 microns, or from about 80 to about 120 microns, or from about 90 to about 100 microns.

In any embodiment described in this specification, a deamidated pea protein isolate swells less in the presence of water and has lower numbers of large particles than non-deamidated pea proteins isolates. In any embodiment described in this specification, a deamidated pea protein isolate comprises a set of particles that, when measured for size in an aqueous slurry, has a particle size distribution, the particle size distribution comprising a set of particles having a size between 150 and 300 microns, wherein a peak number of particles between 150 and 300 microns is less than about 30, or less than about 25, or less than about 20, or less than about 15. Within this specification, a particle size measurement useful for determining the peak number of particles having size between 150 and 300 microns is as follows: a slurry of pea protein isolate in water (5% solids in water w/w) is continuously mixed over period of at least 10 minutes, and the particle size distribution is measured using focused beam reflectance.

In any embodiment described in this specification, a deamidated pea protein isolate has a set of particles that, when measured for size in a aqueous slurry, have a particle size distribution, the particle size distribution comprising a set of particle having a size between 150 and 300 microns, wherein the slurry is measured to have less than about 15 particles having a size between 150 and 300 microns, or less than about 10. Within this specification, a particle size measurement useful for determining the number of particles having size between 150 and 300 microns in slurry is measured in a process consisting of adding pea protein isolate 10 water to form a slurry having 5% solids (w/w) and measuring the particle size distribution immediately following adding the protein.

In any embodiment described in this specification, a deamidated pea protein isolate has an equal or an improved solubility index compared to a fresh sample of a deamidated pea protein isolate. In any embodiment described in this specification, a deamidated pea protein is made by a process comprising storing the deamidated pea protein isolate for at least about 1 month, or at least about 3 months, or at least about 6 months, or at least about 1 year. In any embodiment described in this specification, a deamidated pea protein isolate is obtainable by a process comprising storing the deamidated pea protein isolate at a temperature of from about 30° to about 50° C., or from about 35° C. to about 45° C., or from about 38° C. to about 42° C. for a time of from about 15 to about 45 days, or from about 20 to about 40 days, or from about 25 to about 35 days, or about one month. In any embodiment described in this specification, a deamidated pea protein isolate made by a process comprising storing the deamidated pea protein isolate at a temperature of from about 30° to about 50° C., or from about 35° C. to about 45° C., or from about 38° C. to about 42° C. for a time of from about 15 to about 45 days, or from about 20 to about 40 days, or from about 25 to about 35 days, or about one month.

In any embodiment of a deamidated pea protein isolate described in this specification, the deamidated pea protein isolate has a degree of hydrolysis of less than 10%, or less than 5%, or essentially 0% as measured by SDS-PAGE comparing deamidated and non-deamidated pea protein isolate.

In another aspect, the technology disclosed in this specification pertains to the use a deamidated pea protein isolate in a food composition. In any embodiment, a food composition as disclosed in this specification comprises a deamidated pea protein isolate and a second edible ingredient. In any embodiment, a food composition is a powdered composition comprising a deamidated pea protein isolated and a second powdered ingredient. In any embodiment, a food composition has at least about 15% deamidated pea protein isolate (w/w of the powder), or at least about 25% or at least about 50%, or at least about 60%, or at least about 70%.

In any embodiment, a food composition comprising a deamidated pea protein isolate described in this specification further comprises any second ingredient commonly used in food compositions. In any embodiment, of a food composition described in this specification includes a starch including but not limited to corn starch, tapioca starch, pea starch, fava bean starch, lentil starch, chickpea starch, tapioca starch, potato starch, and sago starch as well as high amylose and low amylose variants of such starches. Such starches also may be within flours and meals including wheat flour and nut meals. Useful starches may be modified or unmodified. Modified starches may be crosslinked including by using phosphate or adipate, or may be stabilized, including hydroxypropylated and acetylated. Useful starch may be converted or hydrolyzed using shear, enzyme, acid, or oxidation. Starch may also be modified usefully by oxidation for purposes other than hydrolysis. Useful starch may be physically modified such as by thermal inhibition, annealing, or heat moisture treatments. Modified and unmodified starch may be pregelatinized or otherwise made cold water soluble.

In any embodiment, a food composition comprising a deamidated pea protein isolate described in this specification may further comprise a sweetener. Useful sweeteners include honey, allulose, tagatose, fructose, glycerol, sucrose, rebaudiosides (A, B, J, M, etc.), and glucosylated stevia glycosides, corn syrups including high fructose corn syrups. Sweeteners may be provided in solid, or powdered, or liquid, or syrup form.

In any embodiment, a food composition comprising a deamidated pea protein isolate described in this specification may further comprise a fiber. Useful fibers may include cellulosic fibers from any botanical source, resistant starches, soluble fibers such as polydextrose or short chain fructooligosacchardies.

In any embodiment, a food composition comprising a deamidated pea protein isolate described in this specification may further comprise a gum or gum-like material. Useful gums and gum like materials include gelling starches, gum Arabic, xanthan gum, tara gum, konjac, carrageenan, locust bean gum, gellan gum, guar gum, pectin, and modified celluloses like carboxymethyl cellulose, and mixtures thereof.

In any embodiment, a food composition comprising a deamidate pea protein isolate described in this specification may further comprise an oil, or fat, or aqueous ingredient. Useful oils include vegetable oils such as corn oil, olive oil, canola oil, sunflower oil, rapeseed oil, palm oil, coconut oil. Useful fats (other than vegetable oils) included animal fats and dairy fats. Useful aqueous ingredients include water, milk, syrups, or other carbohydrate containing liquids, or acidic liquids, or basic liquids.

In any embodiment, a food composition comprising a deamidated pea protein isolate described in this specification may further comprises various other flavorings and coloring commonly used in food composition.

In any embodiment, a food composition, as described in this specification, is a beverage or a cold pressed bar, or powder useful for making a beverage.

In any embodiment, this specification discloses a food composition that is a beverage comprising a deamidated pea protein isolate and an aqueous liquid. In any embodiment, a beverage, as disclosed in this specification is made from a powdered mix comprising a deamidated pea protein isolate. In any embodiment, this specification discloses a beverage comprising a deamidated pea protein isolate and has a pH of less than about 7, or from about 7 to about 2. In any embodiment, this specification discloses a beverage having a pH from about 5 to about 7, or from about 6 to about 7. In any embodiment, this specification discloses a beverage comprising a deamidated pea protein isolate and has a pH of from about 2 to about 4, or about 2 to about 3.5, or from about 2 to about 3.

In any embodiment, this specification discloses a beverage comprising a deamidated pea protein isolate made by a process comprising mixing an aqueous liquid and a powdered composition. In any embodiment, this specification discloses a beverage comprising a deamidated pea protein isolate made by a process comprising mixing an aqueous liquid with a powdered composition having a deamidated pea protein isolate and a second soluble ingredient. In any embodiment, this specification discloses a beverage made from a powdered composition comprising a deamidated pea protein isolate in an amount of at least about 25%, or at least about 50%, or at least about 60%, or at least about 70%, or from about 70% to about 80%. In any embodiment disclosed in this specification, a beverage made from a powdered composition comprising a deamidated pea protein isolate has a protein content of at least about 25%, or at least about 50%, or at least about 60%, or at least about 70%. In any embodiment disclosed in this specification, a beverage made from a powdered composition comprising a deamidated pea protein isolate has a protein content of at least about 25%, or at least about 50%, or at least about 60%, or at least about 70% wherein the protein comes solely from a deamidated pea protein isolate.

In any embodiment, this specification discloses a beverage made by mixing a powder comprising a deamidated pea protein and an aqueous liquid has a peak number of particles having size between 150 and 300 microns that is less than about 30, or less than about 25, or less than about 20, or less than about 15.

In any embodiment, this specification describes a powdered mix for making a beverage wherein a test beverage consisting of 15% (w/w) of the powdered mix in water has a peak number of particles having size between 150 and 300 microns that is less than about 30, or less than about 25, or less than about 20, or less than about 15; wherein the peak particle size is measured using focused beam reflectance while continuously mixing the beverage for at least 10 minutes.

In any embodiment, this specification describes a powdered mix for making a beverage wherein the mix has a soluble material content of at least about 20%, or at least about 25%, or at least about 30% or at least about 35% of the powdered composition is soluble in the aqueous liquid.

In any embodiment, this specification describes a cold pressed food bar comprising a deamidated pea protein isolate and an aqueous ingredient wherein, the cold pressed food bar has a solids content of greater than about 60% (w/w of the bar) or greater than about 70%, from about 60% to about 95%. In any embodiment, an aqueous ingredient for using in a cold pressed food bar, as described in this specification is binding agent, and may be syrup, or other carbohydrate containing liquid. In any embodiment, this specification describes a cold pressed food bar having deamidated pea protein isolate in an amount from 15% to about 50% (w/w of the bar), or from about 15% to about 40%, or from about 15% to about 30%, or from about 20% to about 30%.

In any embodiment, this specification describes a cold pressed food bar comprising a deamidated pea protein isolate that has been deamidated to a degree to control the solubility of the pea protein isolate. In any embodiment described in this specification, a deamidated pea protein isolate for use in a cold pressed food bar has a degree of deamidation of from between about 10% and about 15%, or about 12% and about 14%. In any embodiment described in this specification, a deamidated pea protein isolate for use in a cold pressed food bar has a degree of deamidation of between about 17% and about 23%, or between about 19% and about 21%. In any embodiment described in this specification, a cold pressed food bar has a soluble material content at about pH 7 of between about 30% and about 36%, or about 33% and about 36 (wt%).

In any embodiment a deamidated protein comprises an aqueous ingredient, for example a syrup in an amount of from about 25% to about 35% by weight of the bar.

In any embodiment, this specification describes a cold pressed food bar comprising a deamidated pea protein isolate wherein the degree of deamidation is controlled to reduce the hardness of the cold pressed food bar over time. In any embodiment, a cold pressed food bar described in this specification has a mechanical hardness of less than about 2000 g, or less than about 1500 g. In any embodiment, a cold pressed food bar described in this specification has a mechanical hardness of less than about 2000 g, or less than about 1500 g after at least 1 month’s storage, or 2 months’ storage, or at least 3 months’ storage, or at least 6 months’ storage. In any embodiment described in this specification, a cold pressed food bar and has a percent increase in hardness over 1, 2, 3, or 4 months storage at ambient temperature of less than 40%.

The technology disclosed in this specification also pertains to methods of making a deamidated pea protein isolate. Pea protein isolates maybe deamidated using any method to obtain a deamidated pea protein isolate as described in this specification. In any embodiment, a pea protein isolate can be via an acidic reaction using any food grade acid. In any embodiment disclosed in this specification, a method of making a deamidated pea protein isolate comprises mixing pea protein isolate with water to form a slurry having from about 12% to about 18% solids content, or from about 14% to about 16%; heating the slurry to from about 40° to about 60° C., or from about 45° to about 55° C., or from about 48° C. to about 52° C.; mixing with the slurry glutaminase enzyme in amount of from about 0.35% to 0.075% (w/w), or from about 0.25% to about 0.085%, or from about 0.15% to about 0.095%; and reacting glutaminase enzyme with the slurry at a pH from about 6 to about 8, or from about 6 to about 7.5, or from about 6.5 to about 7.5 to obtain the deamidated pea protein isolate.

In any embodiment, this specification methods for controlling the degree of deamidation to a desired level. In any embodiment, this specification discloses a method for making a deamidated pea protein isolate wherein a glutaminase enzyme is reacted with slurry of pea protein isolate for from about 3 hours to about 24 hours. In any embodiment, this specification discloses a method for making a deamidated pea protein isolate wherein a glutaminase enzyme is reacted with a slurry of pea protein isolate from about 3 hours to about 6 hours, or from about 3 hours to about 5 hours. In any embodiment, this specification discloses a method for making a deamidated pea protein isolate wherein a glutaminase enzyme is reacted with a slurry of pea protein isolate from about 7 hours to about 10 hours, or from about 7 hours to about 9 hours. In any embodiment, this specification discloses a method for making a deamidated pea protein isolate wherein a glutaminase enzyme is reacted with a slurry of pea protein isolate from about 11 hours to about 14 hours, or from about 11 hours to about 13 hours. In any embodiment disclosed in this specification, a deamidated pea protein isolate is made by any process described in this specification.

In any embodiment described in this specification, a method of making a deamidated pea protein isolate comprises mixing pea protein composition with water to form a slurry having a solids content of about 12% to about 18%; heating the slurry to from about 40° to about 60° C. or from about 35° C. to about 45° C.; reacting glutaminase enzyme with the slurry at a pH from about 6 to about 8 for up to about 6 hours or from about 3 to about 6 hours before inactivating the enzyme; and d) recovering a dried deamidated pea protein isolate.

Glutaminase enzyme can be deactivated by increasing the temperature of the slurry to at least about 80° C. for at least about 10 minutes. Deamidated pea protein can be recovered from slurry by various drying methods known in the art, including for example spray drying or freeze drying.

The subject matter described in this specification can be better understood with reference to the following definitions and guidance for construing the terms in this specification.

Reference to the term “pea protein isolate” within this specification means a composition that substantially comprises pea protein. Commonly pea protein isolates are obtained from milled pea compositions (e.g. pea flour) and the pea protein within he flour is increased relative to other using methods, such as those described in this specification, by removing other components of the flour, like starch and fiber. In preferred embodiments described in this specification, a pea protein isolate has protein content greater than about 70%, or greater than about 75%, or from about 75% to about 90% or from about 75% to about 85% protein by weight. The rest of the pea protein isolate is comprised substantially of pea starch and pea fiber (cellulosic material).

Reference to the term “deamidated pea protein isolate” in this specification means a pea protein isolate that has been subjected to a process that removes free amine groups from amino acid residues (glutamine residues) in pea proteins of the pea protein isolate. Deamidation may be done using any process known in the art to obtain the degrees of deamidation described in this specification. In some embodiments a deamidated protein is deamidated using acid. In other embodiments a deamidated protein is deamidated using an enzyme, like a glutaminase enzyme.

Reference to the term “degree of deamidation” in this specification measures amount of ammonia released during deamidation reaction compared to the total ammonia of releasable by the protein. Within this specification, degree of deamidation is reported as a percentage. Degree of deamidation can be calculated using any suitable method in the art. A useful test for measuring degree of deamidation follows. Calculated total ammonia released as follows. Mix three volumes of the enzyme reaction slurry with 1 volume of 40% trichloroacetic acid (“TCA”) solution to obtain mixture having a final TCA concentration of 10%. Centrifuged at 4,000 g for 10 min. Free ammonia content in the supernatant is determined using a commercial ammonia analysis kit (Megazyme). Calculated total ammonia obtainable from the unmodified pea protein isolate as follows. Incubate the unmodified pea protein isolate in 2 M H₂SO₄ solution at boiling temperature (about 100° C.) for 2 hours. After the reaction, mixed 3 volumes of the slurry with 1 volume of 40% TCA solution to obtain a mixture having a final TCA concentration of 10%. Centrifuge the mixture at 4,000 g for 10 min. Free ammonia content in the supernatant was determined using the Megazyme ammonia analysis kit. Degree of deamidation equals total ammonia release divided by total ammonia obtainable multiplied by 100.

Within this specification dissolution properties of deamidated protein are analyzed in part by reference to a test that disperses pea protein in a water (5% solids in water w/w), mixes the protein in water using a common low speed stirring device such as a magnetic stirrer suitable for allowing particle size measurements using focused beam reflectance.

Reference to the term “peak number of particles” in this specification refers to largest number of particles observed within all or part a distribution of particles of a composition that is dispersed in water, like a dispersion of pea protein isolate. A useful test for measuring peak particle size of a composition dispersed in water measures the particle size distribution of the composition for least 10 minutes of continuous stirring.

Use of “about” to modify a number is meant to include the number recited plus or minus 10%. Where legally permissible recitation of a value in a claim means about the value. Use of about in a claim or in the specification is not intended to limit the full scope of covered equivalents.

Recitation of the indefinite article “a” or the definite article “the” is meant to mean one or more unless the context clearly dictates otherwise.

While certain embodiments have been illustrated and described, a person with ordinary skill in the art, after reading the foregoing specification, can effect changes, substitutions of equivalents and other types of alterations to the methods, and of the present technology. Each aspect and embodiment described above can also have included or incorporated therewith such variations or aspects as disclosed regarding any or all the other aspects and embodiments.

The present technology is also not to be limited in terms of the aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to methods, conjugates, reagents, compounds, compositions, labeled compounds or biological systems, which can, of course, vary. All methods described herein can be performed in any suitable order unless otherwise indicated herein or otherwise clearly contradicted by context. It is also to be understood that the terminology used herein is for the purpose of describing aspects only and is not intended to be limiting. Thus, it is intended that the specification be considered as exemplary only with the breadth, scope and spirit of the present technology indicated only by the appended claims, definitions therein and any equivalents thereof. No language in the specification should be construed as indicating any non-claimed element as essential.

The embodiments illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the technology. This includes the generic description of the technology with a proviso or negative limitation removing any subject matter from the genus, regardless of whether the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member, and each separate value is incorporated into the specification as if it were individually recited herein.

The technology disclosed in this specification is further described the following illustrative aspects which are not intended to be limiting in any way.

In a first aspect, the technology disclosed in this specification pertains to a deamidated pea protein isolate comprising a degree of deamidation of between about 10% and about 25%, or between about 15% and about 25%, or between 20% and 25% wherein, optionally, the degree of deamidation is selected from the group consisting of a) between about 10% and about 15%, or about 12% and about 14%; b) between about 17% and about 23%, or between about 19% and about 21%; and c) between about 22% and about 25%, or between about 22% and 24%.

In a second aspect, the technology disclosed in this specification pertains to a deamidated protein isolate disclosed in the first aspect further comprising a percent soluble material content at pH 6 of from about 20% to about 30%, or about 21% and about 27% (wt%) wherein, optionally, the soluble material content at pH 6 is selected from the group consisting of a) between about 20% and about 25%, or between about 20% and about 24%, or between about 20% and about 23% (wt%); b) between about 21% and about 24%, or about 22% and about 24% (wt%); c) between about 22% and about 26%, or about 24% and about 26% (wt%); and d) between about 25% and about 30%, or about 26% and about 28% (wt%).

In a third aspect, the technology disclosed in this specification pertains to a deamidated protein isolate disclosed in the first or second aspects further comprising a percent soluble material content at pH 2 of from about 15% to about 30%, or from about 17% to about 28% wherein, optionally, the soluble material content at pH 2 is selected from the group consisting of a) between about 15% and about 20%, or about 17% and 20% (wt%); b) between about 18% and about 24%, or about 18% to about 22% (wt%); c) between about 22% and about 26%, or about 24% and about 26% (wt%); and d) between about 25% and about 30%, or about 26% and about 28% (wt%).

In a fourth aspect, the technology disclosed in this specification pertains to a deamidated protein isolate disclosed in any one of the first to third aspects further comprising a percent soluble material content at about pH 7 of from about 30% to about 40%, or from about 34% to about 38% (wt%) wherein, optionally, soluble material content at about pH 7 is selected from the group consisting of a) between about 30% and about 36%, or about 33% and about 36 (wt%) and b) between about 35% and about 40%, or between about 35% and 37% (wt%).

In a fifth aspect, the technology disclosed in this specification pertains to a deamidated protein isolate disclosed in any one of the first to fourth aspects further comprising a median particle size of between 75 and 125 microns, or from about 80 to about 120 microns, or from about 90 to about 100 microns.

In a sixth aspect, the technology disclosed in this specification pertains to a deamidated protein isolate disclosed in any one of the first to fifth aspects further comprising a set particles that, when measured for size in a slurry, have a particle size distribution, the particle size distribution comprising a set of particle having a size between 150 and 300 microns, wherein a peak number of particles between 150 and 300 microns is less than about 30, or less than about 25, or less than about 20, or less than about 15; and wherein the peak number of particles between 150 and 300 microns in the slurry is measured in a process consisting of adding pea protein isolate in water to form a slurry having 15% solids and measuring the particle size distribution for at least 10 minutes using focused beam reflectance while continuously mixing the slurry during the measuring.

In a seventh aspect, the technology disclosed in this specification further pertains to the deamidated pea protein isolate disclosed in any one of the first to sixth aspects further comprising a set particles that, when measured for size in a slurry, have a particle size distribution, the particle size distribution comprising a set of particle having a size between 150 and 300 microns, wherein the particle size in slurry is measured in a process consisting of adding pea protein isolate in water to form a slurry having 15% solids and measuring the particle size distribution immediately following adding the protein; and wherein the slurry is measured to have less than about 15 particles having a size between 150 and 300 microns, or less than about 10.

In an eighth aspect, the technology disclosed in this specification further pertains to the deamidated pea protein isolate of any one of the first to seventh aspects being made by a process comprising storing the deamidated pea protein isolate for at least about 1 month, or at least about 3 months, or at least about 6 months, or at least about 1 year.

In a ninth aspect, the technology disclosed in this specification further pertains to the deamidated pea protein isolate of any one of the first to eighth aspects being obtainable by a process comprising: storing the deamidated pea protein isolate at a temperature of from about 30° to about 50° C., or from about 35° C. to about 45° C., or from about 38° C. to about 42° C. for a time of from about 15 to about 45 days, or from about 20 to about 40 days, or from about 25 to about 35 days, or about one month.

In a tenth aspect, the technology disclosed in this specification further pertains to the deamidated pea protein isolate of any one of the first to ninth aspects being made by a process comprising: storing the deamidated pea protein isolate at a temperature of from about 30° to about 50° C., or from about 35° C. to about 45° C., or from about 38° C. to about 42° C. for a time of from about 15 to about 45 days, or from about 20 to about 40 days, or from about 25 to about 35 days, or about one month.

In an eleventh aspect, the technology disclosed in this specification further pertains to the deamidated pea protein isolate of any one of the first to tenth aspects being made by a process further comprising deamidating the pea protein isolate using a glutaminase enzyme.

In a twelfth aspect, the technology disclosed in this specification further pertains to the deamidated pea protein isolate of any one of the first to eleventh aspects being made by a process further comprising: a) mixing pea protein isolate with water to form a slurry having from about 12% to about 18%, or from about 14% to about 16%; b) heating the slurry to from about 40° to about 60° C., or from about 45° to about 55° C., or from about 48° C. to about 52° C.; c) mixing with the slurry glutaminase enzyme in amount of from about 0.35% to 0.075% (w/w), or from about 0.25% to about 0.085%, or from about 0.15% to about 0.095%; and d) reacting glutaminase enzyme with the slurry at a pH from about 6 to about 8, or from about 6 to about 7.5, or from about 6.5 to about 7.5 to obtain the deamidated pea protein isolate.

In a thirteenth aspect, the technology disclosed in this specification further pertains to the deamidated pea protein isolate of any one of the first to twelfth aspects made by a process wherein the glutaminase enzyme is reacted with the slurry for from about 3 hours to about 24 hours, wherein, optionally, the glutaminase enzyme is reacted with the slurry for a time selected from the group consisting of: a) from about 3 hours to about 6 hours, or from about 3 hours to about 5 hours; b) from about 7 hours to about 10 hours, or from about 7 hours to about 9 hours; and c) from about 11 hours to about 14 hours, or from about 11 hours to about 13 hours.

In a fourteenth aspect, the technology disclosed in this specification further pertains to the deamidated pea protein isolate of any one of the first to thirteenth aspects having a protein content (w/w) of greater than about 70%, or greater than about 75%, or from about 75% to about 90% or from about 75% to about 85%.

In a fifteenth aspect, the technology disclosed in this specification further pertains to the deamidated pea protein of any one of the first to fourteenth aspect wherein degree of hydrolysis of less than 10%, or less than 5%, or essentially 0%.

In a sixteenth aspect, the technology disclosed in this specification further pertains to a food composition comprising: the deamidated pea protein isolate of any one of claims first to fifteenth aspect and a second edible ingredient.

In a seventeenth aspect, the technology disclosed in this specification further pertains to the food composition of the sixteenth aspect wherein the food composition is a powdered composition comprising a deamidated pea protein isolate as described in any one of the first to fifteenth aspects and a second powdered ingredient wherein, optionally, the powdered composition comprises at least about 25% deamidated pea protein isolate (w/w of the powder), or at least about 50%, or at least about 60%, or at least about 70%.

In an eighteenth aspect, the technology disclosed in this specification further pertains to the food composition of the sixteenth or seventeenth aspects wherein the food composition is a beverage, cold pressed bar, or powdered composition for making a beverage.

In a nineteenth aspect, the technology disclosed in this specification pertains to the food composition of any one of the sixteenth to eighteenth aspects being a beverage comprising: a deamidated pea protein isolate as described in any one of the first to fifteenth aspects and an aqueous liquid.

In a twentieth aspect, the technology disclosed in this specification pertains to the food composition of any one of the sixteenth to nineteenth aspects being a beverage and further comprising a pH of about 7 or less, or from about 7 to about 2 wherein, optionally, the beverage has a pH selected from the group consisting of a) from about 5 to about 7 or from about 6 to about 7; and b) from about 2 to about 4, or about 2 to about 3.5 or from about 2 to about 3.

In a twenty-first aspect, the technology disclosed in this specification pertains to the food composition of any one of the sixteenth to twentieth aspects being a beverage made by a process comprising mixing a) with the aqueous liquid b) a powdered composition having particles, the powdered composition comprising a deamidated pea protein isolate as described in any one of claims 1 to 13 and a second soluble ingredient wherein, optionally, the powdered composition comprises at least about 25% deamidated pea protein isolate (w/w of the powder), or at least about 50%, or at least about 60%, or at least about 70%.

In a twenty-second aspect, the technology disclosed in this specification pertains to the food composition of any one of sixteenth to twenty-first aspects being a powdered composition useful for making a beverage wherein a test beverage comprising the powdered composition in an amount of 15% the beverage (w/w) has a peak number of particles having size between 150 and 300 microns that is less than about 30, or less than about 25, or less than about 20, or less than about 15; and wherein the peak number of particles having size between 150 and 300 microns is measured using focused beam reflectance while continuously mixing the beverage for at least 10 minutes.

In a twenty-third aspect, the technology disclosed in this specification pertains to the food composition of any one of the sixteenth to the twenty-second being a powdered composition useful for making a beverage wherein the powdered composition has at least about 25% or at least about 30% or at least about 35% by weight of the powdered composition being soluble in the aqueous liquid.

In a twenty-fourth aspect, the technology disclosed in this specification pertains to the food composition of any one of the sixteenth to the twenty-third being a cold pressed food bar comprising: a) the deamidated pea protein isolate of any one of claims 1 to 14; b) and aqueous ingredient wherein, the cold pressed food bar has a solids content of greater than about 60% (w/w of the bar) or greater than about 70%, from about 60% to about 95%.

In a twenty-fifth aspect, the technology disclosed in this specification pertains to the food composition of any one of the sixteenth to the twenty-fourth being a cold pressed food bar having deamidated pea protein isolate in an amount from 15% to about 50% (w/w of the bar), or from about 15% to about 40%, or from about 15% to about 30%, or from about 20% to about 30%.

In a twenty-sixth aspect, the technology disclosed in this specification pertains to the food composition of any one of the sixteenth to twenty-fifth aspects being a cold pressed food bar wherein the degree of deamidation is selected from the group consisting of a) between about 10% and about 15%, or about 12% and about 14%; and b) between about 17% and about 23%, or between about 19% and about 21%.

In a twenty-seventh aspect, the technology disclosed in this specification pertains to the food composition of any one of the sixteenth to the twenty-sixth aspects being a cold pressed food bar having a mechanical hardness of less than about 2000 g, or less than about 1500 g wherein, optionally, the cold pressed food bar has a mechanical hardness of less than about 2000 g, or less than about 1500 g after at least 1 month’s storage, or 2 months’ storage, or at least 3 months’ storage, or at least 6 months’ storage.

In a twenty-eighth aspect, the technology disclosed in this specification pertains to the food composition of any one of the sixteenth to twenty-seventh aspects being a cold pressed food bar wherein the deamidated pea protein isolate has a soluble material content at about pH 7 of between about 30% and about 36%, or about 33% and about 36 (wt%).

In a twenty-ninth aspect, the technology disclosed in this specification pertains to the food composition of any one of the sixteenth to twenty-eighth aspects wherein the aqueous ingredient is selected from the group consisting of sugar syrups, maltitol syrups, high fructose corn syrup, honey, glycerin, allulose syrups, rebaudioside syrups, and mixtures thereof.

In a thirtieth aspect, the technology disclosed in the specification pertains to the food composition of any one of the sixteenth to twenty-ninth aspects further comprising further comprising a gum selected from group consisting of gum Arabic, modified gum Arabic, xanthan gum, gellan gum, guar cum, locust bean gum and carrageenan and mixtures thereof.

In a thirty-first aspect, the technology disclosed in the specification pertains to the food composition of any one of the sixteenth to thirtieth aspects further comprising a starch The food composition of any of claims 16 to 30 further comprising a starch wherein optionally the starch is selected from the group consisting of corn starch, tapioca starch, pea starch, rice starch, sago starch, potato starch, high and low amylose variants of the foregoing, and mixtures thereof.

In a thirty-second aspect, the technology disclosed in the specification pertains to the food composition of any one of the sixteenth to thirty-first aspects further comprising a fiber.

In a thirty-third aspect, the technology disclosed in the specification pertains to the a method of making a deamidated pea protein isolate comprising: a) mixing pea protein isolate with water to form a slurry having from about 12% to about 18%, or from about 14% to about 16%; b) heating the slurry to from about 40° to about 60° C., or from about 45° to about 55° C., or from about 48° C. to about 52° C.; c) mixing with the slurry glutaminase enzyme in amount of from about 0.35% to 0.075% (w/w), or from about 0.25% to about 0.085%, or from about 0.15% to about 0.095%; and d) reacting glutaminase enzyme with the slurry at a pH from about 6 to about 8, or from about 6 to about 7.5, or from about 6.5 to about 7.5 to obtain the deamidated pea protein isolate.

In a thirty-fourth aspect, the technology disclosed in the specification pertains to the method of the thirty-third aspects wherein the glutaminase enzyme is reacted with the slurry for from about 3 hours to about 24 hours, wherein, optionally, the glutaminase enzyme is reacted with the slurry for a time selected from the group consisting of: a) from about 3 hours to about 6 hours, or from about 3 hours to about 5 hours; b) from about 7 hours to about 10 hours, or from about 7 hours to about 9 hours; and c) from about 11 hours to about 14 hours, or from about 11 hours to about 13 hours.

In a thirty-fifth aspect, the technology disclosed in this specification pertains to a deamidated pea protein isolate as described in any foregoing aspect having an improved relative dispersibility compared to a non-deamidated pea protein isolate.

In a thirty-sixth aspect, the technology disclosed in this specification pertains to a deamidated pea protein isolate as described in any foregoing claim having improved relative dispersibility compared to a non-deamidated pea protein isolate after 1 month’s, or 2 months’, or 3 months’, or 6 months’, or 12 months’ storage wherein optionally, the deamidated pea protein isolate is stored at a temperature of from about 30° to about 50° C., or from about 35° C. to about 45° C., or from about 38° C. to about 42° C.

In a thirty-seventh aspect, the technology disclosed in this specification pertains to a deamidated pea protein isolate as described in any foregoing claim having an improved relative dispersibility compared to a non-deamidated pea protein isolate when stored at a temperature of from about 30° to about 50° C., or from about 35° C. to about 45° C., or from about 38° C. to about 42° C. for a time of from about 15 to about 45 days, or from about 20 to about 40 days, or from about 25 to about 35 days, or about one month.

In a thirty-eighth aspect, the technology disclosed in this specification pertains to a deamidated pea protein isolate as described in any foregoing claim stored for 1 month, or 2 months, or 3 months, or 6 months, or 12 months wherein optionally, the deamidated pea protein isolate is stored at a temperature of from about 30° to about 50° C., or from about 35° C. to about 45° C., or from about 38° C. to about 42° C. and wherein the deamidated pea protein isolate has an improved relative dispersibility compared to a fresh deamidated pea protein isolate.

In a thirty-ninth aspect, the technology disclosed in this specification pertains to a deamidated pea protein isolate as described in any of the foregoing claims stored at a temperature of from about 30° to about 50° C., or from about 35° C. to about 45° C., or from about 38° C. to about 42° C. for a time of from about 15 to about 45 days, or from about 20 to about 40 days, or from about 25 to about 35 days, or about one month and having an improved relative dispersibility compared to fresh deamidated pea protein isolate.

In a fortieth aspect, the technology disclosed in this specification pertains to a deamidated pea protein isolate as described in any of the foregoing claims having a relative dispersibility at least about 5%, or at least about 10%, or at least about 15%, or from about 5% to about 25%, or from about 10% to about 25%, or from about 15% to about 25%, or from about 20% to about 25% greater than the relative dispersibility of a non-deamidated pea protein isolate.

In a forty-first aspect, the technology disclosed in this specification pertains to a deamidated pea protein isolate comprising a degree of deamidation of between about 10% and about 20%, or about 12% to about 20% or about 15% to about 20%

In a forty-second aspect, the technology disclosed in this specification pertains the deamidated pea protein isolate of the forty-first aspect further comprising a percent soluble material content at pH 6 of from about 20% to about 25%, or between about 20% and about 24%, or between about 20% and about 23% (wt%).

In a forty-third aspect, the technology disclosed in this specification pertains the deamidated pea protein isolate of the forty-first or forty-second aspect further comprising a percent soluble material content at pH 2 of between about 15% and about 20%, or about 17% and 20% (wt%).

In a forty-fourth aspect, the technology disclosed in this specification pertains the deamidated pea protein isolate of any one of the forty-first to forty-third aspects further comprising a set of particles that, when measured for size in a slurry, have a particle size distribution, the particle size distribution comprising a set of particles having a size between 150 and 300 microns, wherein a peak number of particles between 150 and 300 microns is less than about 30, or less than about 25, or less than about 20, or less than about 15; and wherein the peak number of particles between 150 and 300 microns in the slurry is measured in a process consisting of adding pea protein isolate in water to form a slurry having 15% solids and measuring the particle size distribution for at least 10 minutes using focused beam reflectance while continuously mixing the slurry during the measuring.

In a forty-fifth aspect, the technology disclosed in this specification pertains the deamidated pea protein isolate of any one of the forty-first to forty-fourth aspects further comprising a set of particles that, when measured for size in a slurry, have a particle size distribution, the particle size distribution comprising a set of particles having a size between 150 and 300 microns, wherein the particle size in slurry is measured in a process consisting of adding pea protein isolate in water to form a slurry having 15% solids and measuring the particle size distribution immediately following adding the protein; and wherein the slurry is measured to have less than about 15 particles having a size between 150 and 300 microns, or less than about 10.

In a forty-sixth aspect, the technology disclosed in this specification pertains to a deamidated pea protein isolate that when dispersed in water in an amount of 15% solids (wt.%) forms a slurry such that when the slurry is continuously mixed for at least 10 minutes, the slurry has a particle size distribution comprising a set of particles having a size between 150 and 300 microns, wherein a peak number of particles between 150 and 300 microns is less than about 30, or less than about 25, or less than about 20, or less than about 15.

In a forty-seventh aspect, the technology disclosed in this specification pertains to the forty-sixth aspect wherein the particle size distribution immediately following adding the protein to the water is measured to have less than about 15 particles having a size between 150 and 300 microns, or less than about 10.

In a forty-eighth aspect, the technology disclosed in this specification pertains to the forty-sixth or forty-seventh aspects, further having a degree of deamidation a) between about 10% and about 20% or about 12% and 20%, or from about 15% to about 20%; and b) 10% and 17% %, or about 12% to less than 17%, or about 12% to about 16%.

In a forty-ninth aspect, the technology disclosed in this specification pertains the deamidated pea protein isolate of any one of the forty-sixth to forty-eighth aspects further comprising a percent soluble material content at pH 6 of from about 20% to about 25%, or between about 20% and about 24%, or between about 20% and about 23% (wt.%).

In a fiftieth aspect, the technology disclosed in this specification pertains the deamidated pea protein isolate of any one of the forty-sixth to forty-ninth aspects further comprising a percent soluble material content at pH 2 of between about 15% and about 20%, or about 17% and 20% (wt%).

In a fifty-first aspect, the technology disclosed in this specification pertains to a food composition comprising: at least 15% deamidated pea protein isolate of, optionally being the deamidated pea protein isolate as described in any one of the forty-first to fiftieth aspects, (wt.% of the composition) and a second edible ingredient.

In a fifty-second aspect, the technology disclosed in this specification pertains to the food composition of the fifty-first aspect being selected from the group consisting of a beverage, a cold pressed bar, and a powdered composition for making a beverage.

In a fifty-third aspect, the technology disclosed in this specification pertains to the food composition of the fifty-first or fifty-second aspects being a powdered composition and wherein deamidated protein is in an amount from about 70% to about 80% (wt.%).

In a fifty-fourth aspect, the technology disclosed in this specification pertains to the food composition of any one of the fifty-first to fifty-third aspects being a powdered composition capable of forming a beverage by mixing the powdered the powdered in an amount of 15% (wt. of the beverage) with water wherein the beverage when continuously stirred for at least 10 minutes has a particle size distribution having an observed peak number of particles having size between 150 and 300 microns of less than about 30, or less than about 25, or less than about 20, or less than about 15.

In a fifty-fifth aspect, the technology disclosed in this specification pertains to the food composition of any one of the fifty-first to fifty-fourth aspects being a powdered composition useful for making a beverage wherein at least about 20% of protein in the powdered composition (wt.% of the composition) is soluble in aqueous liquid having pH of at least about 6.

In a fifty-sixth aspect, the technology disclosed in this specification pertains to the food composition of any one of the fifty-first to fifty-fifth aspects being a cold pressed food bar a) wherein the deamidated pea protein isolate is in an amount of from 15% to about 30% or from about 20% to about 30% w/w of the bar b) wherein the second ingredient is an aqueous ingredient in an amount of from 25% to 35% (w/w); and c) wherein the composition has a solids content from about 60% to about 95% (wt.%).

In a fifty-seventh aspect, the technology disclosed in this specification pertains to the food composition of any one of the fifty-first to fifty-sixth aspects being a cold pressed food bar and having a percent increase in hardness over 1, 2, 3, or 4 months storage at ambient temperature of less than 40%.

In a fifty-seventh aspect, the technology disclosed in this specification pertains to a method of making a deamidated pea protein isolate comprising: a) mixing pea protein composition with water to form a slurry having a solids content of about 12% to about 18%; b) heating the slurry to from about 40° to about 60° C.; c) reacting glutaminase enzyme with the slurry at a pH from about 6 to about 8 for up to about 6 hours or from about 3 to about 6 hours before inactivating the enzyme; and d) recovering a dried deamidated pea protein isolate.

In a fifty-eighth aspect, the technology disclosed in this specification pertains to the method of the fifty-seventh aspect further comprising recovering and drying the deamidated pea protein isolate and storing the pea protein isolate at a temperature of from about 35° C. to about 45° C.

In a fifty-ninth aspect, the technology disclosed in this specification pertains to the method of the fifty-seventh or fifty-eighth aspects wherein the deamidated protein is as described in any one of claims 1 to 10.

The technology disclosed in this specification is further described by reference to the following examples, which are not limiting in any way.

### EXAMPLE 1 - FORMULATIONS, HIGH PROTEIN INSTANT DRINK MIX

Table 1 describes an illustrative recipe for a high protein instant drink mix. Variations to this formula are possible, including but not limited to the addition of pregelatinized or otherwise soluble starch, other gums, other sweeteners, flavor carriers, for example including, but not limited, to maltodextrin, which may or may not be soluble in water, and various soluble fibers or cellulosic materials.

TABLE 1

High Protein Instant Drink Mix

| Formula | |
|---|---|
| Ingredient | Wt% |
| Deamidated Pea Protein Isolate (about 80% protein content by weight of the isolate) | 78.64 |
| Prebiotic Fiber | 10.48 |
| Cane Sugar Crystals | 2.62 |
| Natural Vanilla Flavor | 3.93 |
| Gum Arabic, Spray Dry Powder | 3.93 |
| Sweetener | 0.39 |
| TOTAL | 100 |

All ingredients are provided as dried powder. Deamidated pea protein isolate is treated with glutaminases as described in this specification and has a protein content of 80% by weight of the isolate. To make the mix all ingredients are sifted into a closable container and are further agitated to uniformly distribute the ingredients in the batch of the high protein instant drink mix.

Drinks are made by mixing 37 g of the prepared high protein instant drink mix to 8oz (about 237 ml) of water or other desired aqueous liquid. Mixture of powder and liquid is stirred or otherwise agitated to disperse or dissolve.

### EXAMPLE 2 - FORMULATION, COLD PRESSED FOOD BAR

Table 2 describes an illustrative recipe for a cold pressed food bar. Variations and additions to this formula are possible consistent with standard practices in the industry. The following formula is scaled for manufacture using standard kitchen equipment, but the scale of ingredients and equipment used can be adjusted as needed.

TABLE 2

Cold Pressed Food Bar

| Ingredient | Wt (g) | wt% |
|---|---|---|
| Maltitol Syrup | 418.05 | 27.87 |
| Honey | 112.8 | 7.52 |
| Canola Oil | 56.4 | 3.76 |
| Glycerin (99.7% wt%) | 28.2 | 1.88 |
| Stevia Leaf Extract | 0.45 | 0.03 |
| Pea Protein Isolate (%db) (80% protein wt%) | 347.85 | 23.19 |
| Salt | 2.85 | 0.19 |
| Almond Meal | 319.35 | 21.29 |
| Cocoa Powder | 214.05 | 14.27 |
| Total | 1500 | 100 |

Deamidated pea protein isolate is treated with glutaminases as described in this specification and has a protein content of 80% by weight of the isolate.

All dry ingredients are mixed except the stevia leaf extract. The honey and maltitol syrup are mixed and heated in a separate container until the mixture reaches 130o F (about 54o C) where it is continuously stirred until the syrup reaches a target Brix of from 78o to 81o Brix. Canola oil and glycerin are then added to the syrup mix, followed by stevia leaf extract, which is mixed until dissolved. Dry blend is then added to the mixture of oil and sweeteners and all ingredients are mixed until homogenous. Bars are formed by hand to desired size.

### EXAMPLE 3 - REACTIONS CONDITIONS FOR MAKING DEAMIDATED PEA PROTEIN ISOLATE

Pea protein isolate (80% protein content) was mixed with water to make a 15% (w/w) slurry. The slurry was pre-heated to 50° C. with stirring. Glutaminase enzyme (PG500 from Amano) was added to the slurry at a dosage of 0.1% (w/w of protein). The reaction occurred at about 50° C. (normally between 45° - 55° C.) and at about pH 7. Reactions were run for 4, 8, 12, 16, and 24 hours. For each reaction, the glutaminase enzyme was deactivated by heating the slurry to 80° C. and holding it at that temperature for 10 minutes. The deamidated protein isolate was recovered by freeze-drying or spray-drying the slurry.

The degree of deamidation was measured for samples of pea protein isolate deamidated with glutaminase enzyme recovered after 0, 4, 8, 12, 16, and 24 hours of reaction time. Three volumes of the enzyme reaction slurry were mixed with 1 volume of 40% trichloroacetic acid (“TCA”) solution to obtain mixture having a final TCA concentration of 10%. The TCA mixture was then centrifuged at 4,000 g for 10 min. Free ammonia content in the supernatant was determined using a commercial ammonia analysis kit (Megazyme). This measurement reflects ammonia released from the deamidation process.

The total ammonia obtainable from the unmodified pea protein isolate was determined by incubating the unmodified pea protein isolate in 2 M H2SO4 solution at boiling temperature (about 100° C.) for 2 hours. After the reaction, 3 volumes of the slurry were mixed with 1 volume of 40% TCA solution to obtain a mixture having a final TCA concentration of 10%. The mixture was then centrifuged at 4,000 g for 10 min. Free ammonia content in the supernatant was determined using the Megazyme ammonia analysis kit. This measurements reflects the total ammonia that can be released from the protein.

Degree of deamidation is calculated as the ammonia released during deamidation reaction divided by the total ammonia of releasable by the protein x 100. The degree of deamidation in samples of pea protein isolate deamidated with glutaminase enzyme for 0, 4, 8, 12, 16, and 24 hours is described in Table 3.

TABLE 3

Degree of deamidation of pea protein samples

| Sample | Reaction Time (hours) | Degree of deamidation (%) |
|---|---|---|
| Sample 1 | 0 | 0.87 |
| Sample 2 | 4 | 12.99 |
| Sample 3 | 8 | 20.10 |
| Sample 4 | 12 | 23.38 |
| Sample 5 | 16 | 24.36 |
| Sample 6 | 24 | 25.35 |

As seen, degree of deamidation substantially plateaus between 8 and 12 hours of reaction time, with relatively minimal increases in deamidation seen after 12 hours of reaction time.

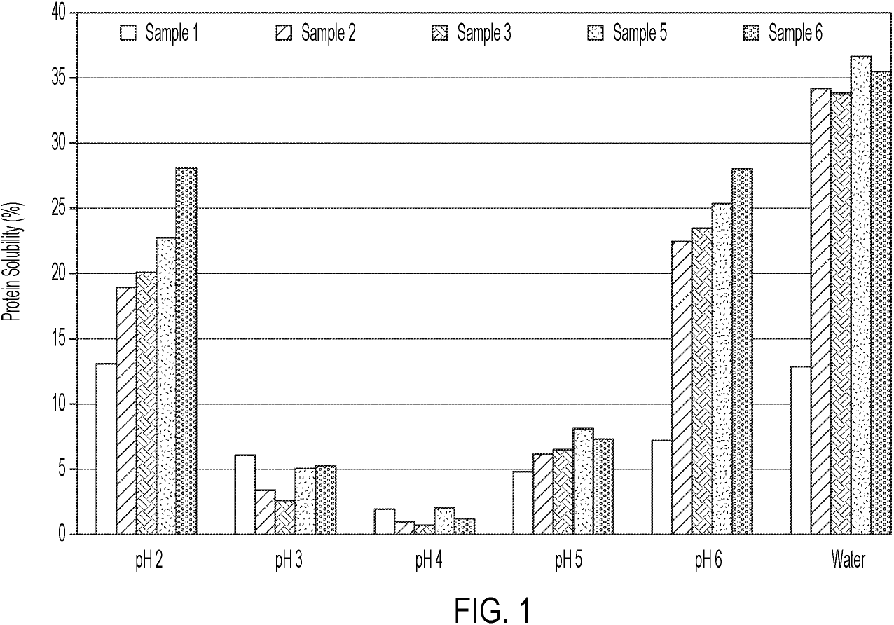

FIG. 1 shows the relative solubility of Samples 1, 2, 3, 5, and 6 at pH 2, 3, 4, 5, 6, and in unmodified water. As seen, pea protein isolate solubility is affected by pH, with all samples being least soluble at pH 4. Otherwise, solubility generally tracks deamidation with more highly deamidated pea protein isolates being more soluble than less deamidated pea protein isolate.

Percent soluble material content is measured using Bradford method, which is a colorimetric protein assay based on an absorbance shift of the dye Coomassie Blue. The higher soluble material content (the more of a sample that is soluble), the more binding of the dye occurs, giving more color/absorbance shift, which is measured by a spectrophotometer.

### EXAMPLE 4 - EFFECT OF DEAMIDATION ON LONG TERM DISSOLUTION PROPERTIES

The dissolution properties of pea protein isolate deamidated with glutaminase enzyme was measured with unmodified pea protein isolates being prepared in two ways. The dissolution analysis determined particle size distributions for various pea protein dispersed in water (5% solids in water w/w) the isolate is continuously mixed for the duration of the test and the particle size distribution is measured using focused beam reflectance.

Sample A - is a spay dried version of a commercially available pea protein isolate available from Ingredion Incorporated. In its commercial form the material is a powder having pea protein content of about 80% (wt%) and has a median particle size of around 100 microns. Spray drying did not affect measured protein content which remained at about 80% (wt%). Spray drying was used to establish a relatively constant particle size among samples

Sample B - is pea protein isolate deamidated with glutaminase enzyme. The starting material is the sample commercial sample used in Sample A. Sample A was then mixed with water to make a 15% (w/w) slurry. The slurry was pre-heated to 50° C. with stirring. Glutaminase enzyme (PG500 from Amano) was added to the slurry at a dosage of 0.1% (w/w of protein). The reaction occurred at about 50° C. and was run for 16 hours. The deamidation reaction did not affect the measured protein content, which remained at about 80%. The deamidated protein isolate was spray dried to match the median particle size of Sample A.

Samples A and B were stored at 40° C. for 1 month after which dissolution properties of fresh and stored samples were measured. Dissolution measurements were obtained by mixing protein powder with water to obtain 5% solids, and continuously mixing the slurry for 30 minutes at room temperature. Dissolution properties were determined by monitoring the changes in particle size distribution of the protein in water using focused beam reflectance measurement. The Samples of A and B stored at 40° C. for one month are labeled Samples A′ and B′

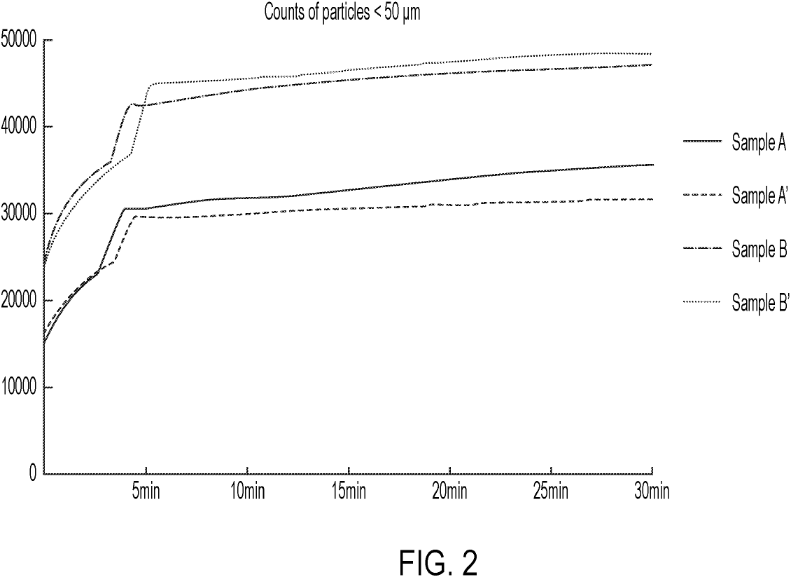

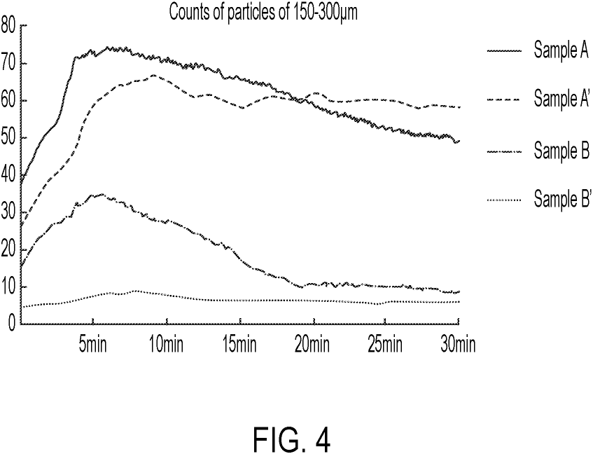

Dissolution properties of Sample A, and A′ and B and B′ are reported in FIGS. 2 and 4. FIG. 4 reports the changes in particles having size between 150 and 300 microns. FIG. 2 reports the increase in particles having size less than 50 micron over mixing. With reference to FIG. 4, it is seen that for both Sample A and A′ and Sample B, particle size increases for a time and then decreases. It is believed that the observation shows that the protein swells as it rehydrates and then breaks down to first disperse and ultimately dissolve into solution.

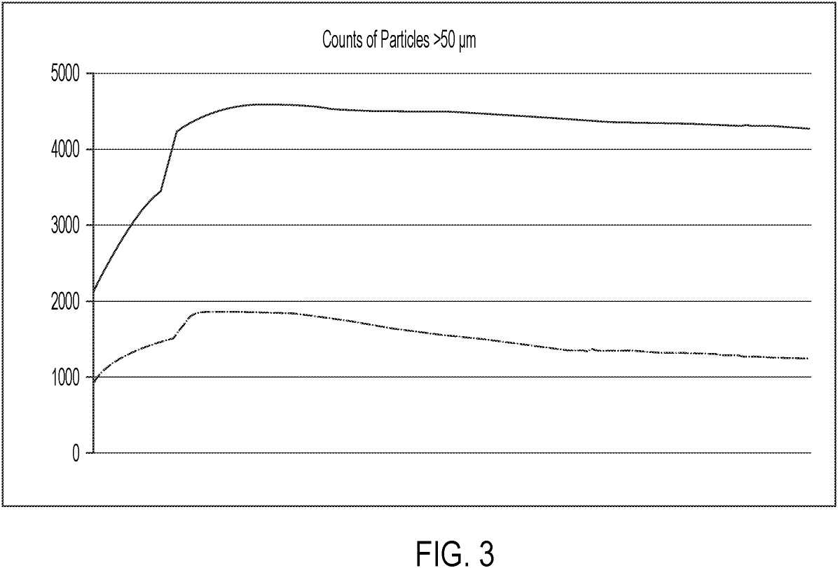

Sample B′, as shown in FIG. 3, has a different dissolution profile, having significantly less large protein particles throughout the dissolution process. It is believed that this shows that the deamidated pea protein isolate after 1-month at 40° C. storage swells less than either Sample B or either sample of Samples A or A′.

With reference to FIG. 2, for Sample B and B′, the dissolution properties, as indicated by the number of small particles (< 50 microns) released while being mixed in water, is similar for both the fresh sample and the sample after 1-month storage at 40° C., although the stored sample releases slightly more small particles (< 50 microns) than the fresh sample. On the other hand, with reference to Samples A and A′ is mixed in water, much fewer small particles (< 50 microns) are released from the stored samples than that from the fresh sample, indicating deteriorated dissolution properties after storage.

FIG. 3 depicts the distribution of particles greater than 50 microns and shows similar dissolution profiles for of Sample A versus Sample B as seen in FIGS. 2 and 3 (stored samples are not reported). For example, Sample A has more particles having size above 50 microns than Sample B, which is consistent with Sample A having both more particles having size from 150 to 300 microns (FIG. 4) and less particles having size less than 50 microns (FIG. 2). Also, FIG. 3 shows the increase in particles early during mixing followed by a decrease in particles as mixing continues like what is seen in FIG. 4.

The results shown in FIGS. 2 and 4 show that samples of deamidated pea protein isolate stored at 40o C for one month have the same dissolution properties, if not better, compared to fresh samples. In contrast, the profile of Sample B shows that dissolution rate decreases over time.

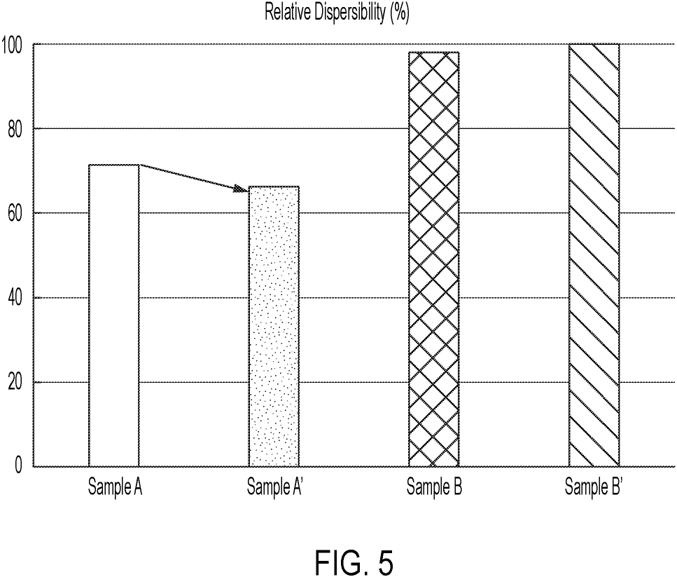

Results shown in FIGS. 2 and 4 are further supported by the results of FIG. 5, which depicts the relative dispersibility of Samples A and B by comparing the dispersibility of fresh samples Samples A and B and Samples A′ and B′ stored at 40° C. for 1-month. Dispersibility is measured as the number of small particles (< 50 microns) released from the protein samples after being mixed in water for 30 mins. The relative dispersibility plotted here sets at 100% the most dispersed sample after being mixed in water for 30 minutes and then compares the dispersibility of the other samples relative to the most dispersed.

As seen, Sample B′ held for 1 month at 40° C. is set to a relative dispersibility of 100%. Sample A′ is the least dispersed sample. Sample B differs from sample A because it has much higher dispersibility overall. The relative dispersibility of Sample B to Sample B′ decreased from ~72% in the fresh sample to about 65% over stored for 1 month at 40° C., again suggesting the deteriorated dispersibility after storage. On the other hand, for Sample B′, the stored sample, exhibits the same or even slightly better dispersibility than the fresh sample, Sample B.

### EXAMPLE 5 - SENSORY EFFECT OF DEAMIDATION: PEA PROTEIN SUSPENDED IN WATER

Sensory properties of deamidated pea protein isolate (80% protein) were measured by a seven-person employee panel. Panelists were trained during two train sessions prior to the evaluation where they (i) identified basic tastes & generation of attributes practice with flour and water solutions, and (ii) Comparative Analysis practice with market RTD beverages according to the ten attributes to be measured. The attributes measured were (i) overall attributes of aroma intensity, appearance of the suspension; (ii) mouth-feel attributes of overall flavor intensity, sweet taste, bitter taste, salt taste, thickness, smoothness, and (iii) residual attributes of overall aftertaste and mouth drying.

Evaluation method was a comparative analysis that compared four samples of dispersions of pea protein isolate (6.6% by weight) in water. The pea protein isolates used were made according to the methods used to make Samples 1, 2, 3, and 4. Deamidated pea protein isolated made as described for Samples 5 and 6 were excluded because prior solubility analysis showed that despite minor increases in deamidation, solubility of the product did not increase measurably following 12 hours of reaction time (Sample 4).

Dispersions of pea protein were made on site on the day of testing by mixing pea protein isolate and water. Dispersion Sample I was made using pea protein isolate Sample 1. Dispersion Sample II was made using pea protein isolate Sample 2. Dispersion sample III was made using pea protein isolate Sample 3. Dispersion Sample IV was made using pea protein isolate Sample 4. Mixed samples were stored and evaluated at 70o F (about 21° C.). Samples were provided to panelists in 2 fluid ounce clear plastic serving cups (about 28 ml).

Dispersion Samples II, III, and IV were compared with Dispersion Sample I for the 10 attributes. Results were recorded on paper ballots. Dispersion Sample I was defined to have a rating of zero and Dispersion Samples II, III, and IV were scored to measure the difference of an attributed compared to Dispersion Sample I on a scale of +5 to -5. Positive values indicated that an attribute was more intense in the Sample compared to Dispersion Sample I, and a negative value indicated that the attribute was less intense in the Sample compared to Dispersion Sample I.

Samples were presented in numerical order with one replicate. More specifically, panelists evaluated Dispersion Sample I for reference then compared to Dispersion Sample II, cleansed their palate, then repeated this evaluation procedure for Dispersion Sample III and IV. For palate cleansers, panelists were provided spring water and unsalted cracker.

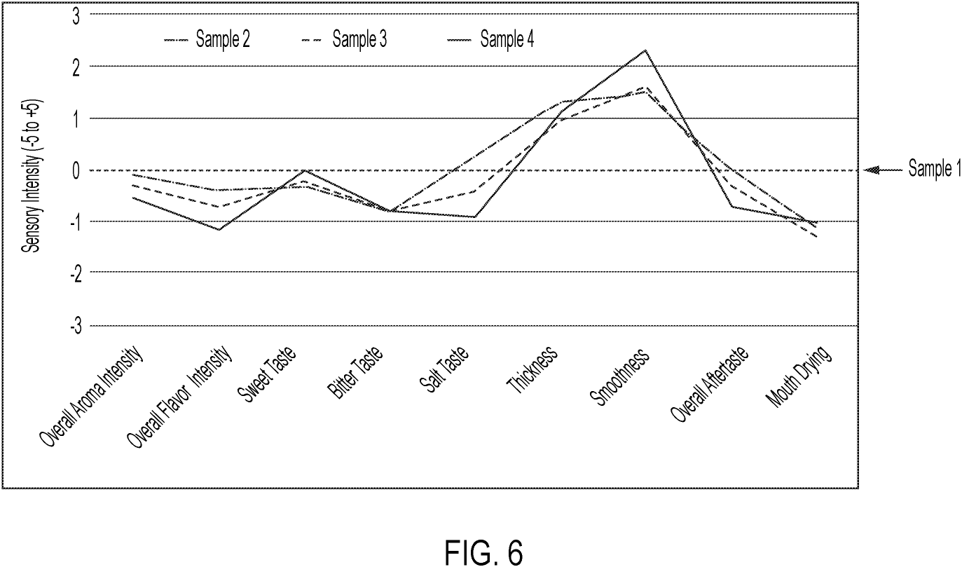

Data from ballots was analyzed using XLSTAT® (v2019), statistical analysis software, and is reported in FIG. 6. Starred attributes were found to differ significantly from the unmodified sample (Dispersion Sample I) at a confidence level of 95%. As seen, four attributes were determined to have statistically significant difference from Dispersion Sample I, overall flavor, thickness, smoothness, and mouth dryness.

As seen in FIG. 6, all samples of dispersed deamidated pea protein have more smoothness and less chalkiness and less mouth dryness, than the samples of dispersed non-deamidated protein. It is also seen that Dispersion Sample IV, which was treated with glutaminase for the longest time, was rated the smoothest sample. This suggests that the high dispersibility of the deamidated pea protein relates to smoothness in dispersed samples, perhaps because of the reduced overall particle size of the deamidated pea protein isolate compared to the non-deamidated pea protein isolate.

### EXAMPLE 6 - SENSORY EFFECT OF DEAMIDATION TIME: PEA PROTEIN SUSPENDED IN WATER

Initial evaluations reported in Example 5 found that although texture and suspension times improved with deamidation, undesirable flavors of fermented vegetation and rotting along with undesirable aromas of musty increased with reaction time. Second evaluation was undertaken to evaluate more exactly onset of increased fermented vegetation/rotting flavors. Evaluation was like that described in Example 5

Four dispersions were made comprising pea protein isolate deamidated using glutaminase enzyme (PG500 from Amano) at about 50° C. dispersed in water for 0, 4, 6, or 8 hours. Samples were dispersions of pea protein isolate in water at a solids content of 6.6% (wt.%, accounting for a 5% moisture content of samples). Samples were stirred for about 45 seconds and stored at ambient temperatures (about 21° C.) for use within eight hours of mixing. Samples were presented in 2 ounce (about 59 ml) transparent plastic cups with lids. Samples were presented to eight trained panelists in monadic and randomized order. Panelists evaluate samples on a comparative scale against a reference sample (0 hours of deamidation). Samples were scored based on difference from the reference on a scale of -5 to +5 based on the relative intensity difference of an attribute between a sample and the reference sample. Reference samples had attribute scores of 0. Positive scores indicate an attribute was more intensely observed in a sample than in the reference. Negative scores indicate that an attribute was less intensely in a sample than in the reference. Samples were present at intervals of five minutes and panelists were provided heavy cream and spring water palate cleansers. Data was entered in Compusense®, Cloud software and statistical analysis of the data was done using XLSTAT (v2019).

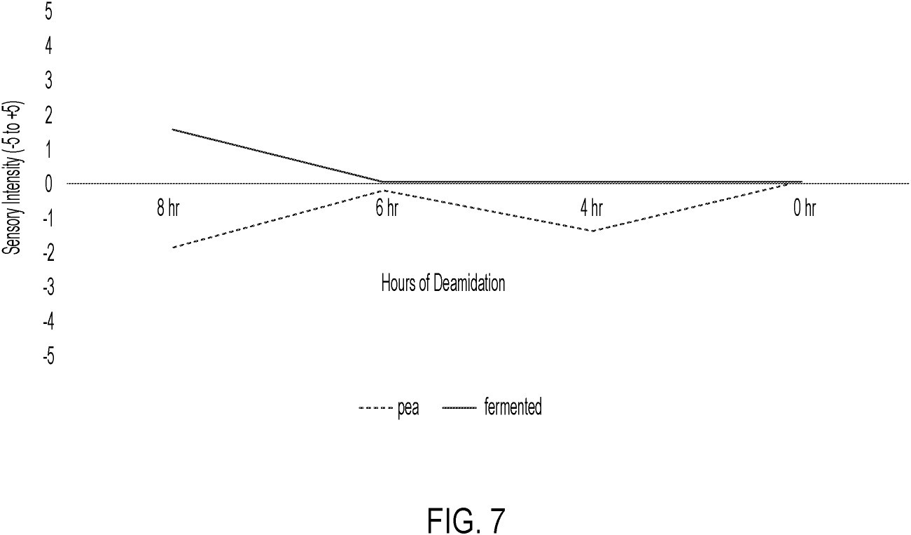

Results are reported in FIG. 7. As seen, sample had increased fermented flavor after 6 hours of fermentation.

### EXAMPLE 7 - POWDERED BEVERAGES

Pea protein isolate dispersions of Example 6 were also evaluated for separation by trained panelists. Panelists evaluated the appearance and noted the amount of visible separation of the pea protein isolate from water. Samples were scored based on difference from the reference on a scale of -5 to +5 based on the relative intensity difference of an attribute between a sample and the reference sample. Reference samples had attribute scores of 0. Positive scores indicate that a sample was more suspended (less separated) than the reference. Negative scores indicate that a sample was less suspended (more separated) than the reference. All samples, whether, deamidated for 4, 6 or 8 hours were significantly more suspended than the references sample but were not observed to have significant difference relative to each other.

### EXAMPLE 8 - COLD PRESSED FOOD BARS

Deamidated pea proteins make them useful cold pressed food bars because they do not form agglomerates that adversely affect the texture of the bar, for example by increasing a mouth drying sensation or by making a less smooth bar. Further the reduced tendency of deamidated pea protein isolate stored for 1 month at 40° C. to form larger agglomerates makes them particularly suitable for use in cold pressed food bars.

This further seen because cold pressed bar using deaminated pea protein isolate were softer than cold pressed bars using non-deamidated pea protein isolate. Cold pressed bars were made using the formula of Table 2 and the associated method using either a deamidated protein or a non-deamidated protein. Deamidated protein was made by deamidating pea protein isolate (80% protein w/w) for 6 hours at about 50° C. using glutamines enzyme (PG500 from Amano).

Bars were stored at ambient temperatures and were measured for hardness after 1, 2, 3, and 4 weeks storage. Hardness of the protein bars was measured using a Texture Analyzer. Probe TA-8A (round end probe). Testing speed was 1 mm/sec, and trigger force was 3 grams. Travel distance into the bar was 5.5 mm. Cold pressed bars made using deamidated pea protein isolate had a percent increase in hardness over 4 weeks storage time of less than about 40% and had smaller percent increase in hardness than non-deamidated pea protein isolate at ever storage interval. Measured hardness and percent increase of hardness over time are listed reported in Table 4.

TABLE 4

Hardness of Cold Pressed Bar

| | Regular Protein | | Deamidated Protein | |
|---|---|---|---|---|
| | Hardness (g) | Increase% | Hardness (g) | Increase% |
| Week 0 | 1453.7 | | 2236.9 | |
| Week 1 | 1896.2 | 30.4 | 2438.0 | 9.0 |
| Week 2 | 2262.2 | 55.6 | 2559.9 | 14.4 |
| Week 3 | 2709.4 | 86.4 | 2818.4 | 26.0 |
| Week 4 | 3347.0 | 130.2 | 3066.8 | 37.1 |

What is claimed:

1. A deamidated pea protein isolate comprising a degree of deamidation of between about 10% and 17%.

2. The deamidated pea protein isolate of claim 1 further comprising a percent soluble material content at pH 6 of from about 20% to about 25%, (wt%).

3. The deamidated pea protein isolate of claim 1 further comprising a percent soluble material content at pH 2 of between about 15% and about 20%, (wt%).

4. The deamidated pea protein isolate of claim 1
further comprising a set of particles that, when measured for size in a slurry, have a particle size distribution, the particle size distribution comprising a set of particles having a size between 150 and 300 microns, wherein a peak number of particles between 150 and 300 microns is less than about 30,
wherein the peak number of particles between 150 and 300 microns in the slurry is measured in a process consisting of adding pea protein isolate in water to form a slurry having 15% solids and measuring the particle size distribution for at least 10 minutes using focused beam reflectance while continuously mixing the slurry during the measuring.

5. The deamidated pea protein isolate of claim 1
further comprising a set of particles that, when measured for size in a slurry, have a particle size distribution, the particle size distribution comprising a set of particles having a size between 150 and 300 microns,
wherein the particle size in slurry is measured in a process consisting of adding pea protein isolate in water to form a slurry having 15% solids and measuring the particle size distribution immediately following adding the protein; and
wherein the slurry is measured to have less than about 15 particles having a size between 150 and 300 microns.

6. A deamidated pea protein isolate that when dispersed in water in an amount of 15% solids (wt.%) forms a slurry such that when the slurry is continuously mixed for at least 10 minutes, the slurry has a particle size distribution comprising a set of particles having a size between 150 and 300 microns, wherein a peak number of particles between 150 and 300 microns is less than about 30.

7. The deamidated pea protein isolate of claim 6 wherein the particle size distribution immediately following adding the protein to the water is measured to have less than about 15 particles having a size between 150 and 300 microns.

8. The deamidated pea protein isolate of claim 6 further having a degree of deamidation between 10% and 17%.

9. The deamidated pea protein isolate of claim 6 further comprising a percent soluble material content at pH 6 of from about 20% to about 25%, (wt%).

10. The deamidated pea protein isolate of claim 6 further comprising a percent soluble material content at pH 2 of between about 15% and about 20%(wt%).

11. A food composition comprising: the at least 15% deamidated pea protein isolate (wt.% of the composition) and a second edible ingredient; wherein the pea protein isolate has a degree of deamidation of between about 10% and 17%.

12. The food composition of claim 11 being selected from the group consisting of a beverage, a cold pressed bar, and a powdered composition for making a beverage.

13. The food composition of claim 11 being a powdered composition and wherein deamidated pea _protein is in an amount from about 70% to about 80% (wt.%).

14. The food composition of claim 11 being a powdered composition capable of forming a beverage by mixing the powdered composition in an amount of 15% (wt. of the beverage) with water
wherein the beverage when continuously stirred for at least 10 minutes has a particle size distribution having an observed peak number of particles having size between 150 and 300 microns of less than about 30.

15. The food composition of claim 11 being a powdered composition useful for making a beverage wherein at least about 20% of protein in the powdered composition (wt.% of the composition) is soluble in aqueous liquid having pH of at least about 6.

16. The food composition of claim 11 being a cold pressed food bar
wherein the deamidated pea protein isolate is in an amount of from 15% to about 30% w/w of the bar
wherein the second ingredient is an aqueous ingredient in an amount of from 25% to 35% (w/w); and
wherein the composition has a solids content from about 60% to about 95% (wt.%).

17. The food composition of claim 11 being a cold pressed food bar and having a percent increase in hardness over 1-, 2-, 3-, or 4-months storage at ambient temperature of less than 40%.

18. A method of making a deamidated pea protein isolate comprising:
a) mixing a pea protein composition with water and a glutaminase enzyme to form a slurry having a solids content of about 12% to about 18%, and allowing the glutaminase enzyme to react with the pea protein for a time up to about 6 hours before inactivating the enzyme; and
b) recovering a dried deamidated pea protein isolate
wherein, the glutaminase enzyme is in an amount or from about 0.15% to about 0.095% (of the slurry); and
wherein, the pH of the slurry is between about 6 and about 8.

19. The method of claim 18 further comprising dry heating the dried deamidated pea protein isolate at temperature of from about 35° C. to about 45° C.

20. The method of claim 18 wherein the deamidated protein has a degree of deamidation of between about 10% and 17%.
